(19) United States
(12) Patent Application Publication
(10) Pub. No.: US 2025/0111800 A1
(43) Pub. Date: Apr. 3, 2025

(54) Methods for conducting memory therapy using facial recognition and vibrotactile stimulation with synchronized music

(71) Applicant: Mark Freeman, Scottsdale, AZ (US)

(72) Inventor: Mark Freeman, Scottsdale, AZ (US)

(21) Appl. No.: 18/479,192

(22) Filed: Oct. 2, 2023

(51) Int. Cl. G09B 19/00; G06F 3/01; G06F 3/04883; G06N 3/091; G09B 21/00; G16H 20/70; G99Z 99/00

(52) U.S. Cl. CPC G09B 19/0023; G06F 3/016; G06F 3/04883; G06N 3/091; G09B 21/007; G16H 20/70; G06F 2203/014

(57) ABSTRACT

Methods of the present disclosure are designed to conduct memory therapy using facial recognition and vibrotactile stimulation with synchronized music. A haptic pattern generates vibrotactile stimulation on a mobile computing device for the targeted activation of sensory neurons in fingertips, causing an increase of dopamine levels in the brain and improving the efficacy of memory therapy. Synchronization of the haptic pattern with music produced by speakers within the mobile computing device or external headphones, creates a synergistic effect that further increases dopamine levels to enhance memory function. A facial recognition algorithm analyzes digital images and videos to identify familiar individuals by name and relationship. The algorithm utilizes the facial recognition data to generate personalized cognitive challenges, and continually adjusts the difficulty level to ensure sustained user engagement. The vibrotactile stimulation with synchronized music reinforces learning and recall during cognitive challenges to increase the efficacy of memory therapy.

### BACKGROUND OF THE INVENTION

Cognitive decline affects millions of people and causes the loss of memory function to such an extent that it interferes with a person's daily life and activities. A non-invasive and non-pharmacological method for conducting memory therapy could provide significant benefits for people with cognitive decline.

Tactile neurons in human fingertips detect the vibrations of touch sensation, and convey signals to the brain for a textural representation of the physical world. Research studies in neuroscience have shown that vibrotactile stimulation of sensory neurons in the fingertips at specific frequencies increases the level of dopamine in the hippocampus, which can improve memory function, spatial learning and synaptic plasticity. Devices typically used during clinical studies to generate vibrotactile stimulation are cumbersome and bulky. A method for generating vibrotactile stimulation on a mobile computing device using a specialized haptic pattern could provide significant benefits for people with cognitive decline. Synchronizing the vibrotactile stimulation with music creates a synergistic effect that further increases dopamine levels in the brain, improving the efficacy of memory therapy using multi-sensory stimulation.

Existing methods for strengthening memory function are limited to games and puzzles that use words, numbers and generic images. A personalized method that uses facial recognition to challenge users for identifying loved ones in photographs and videos would provide significant benefits for reinforcing memory function.

Since each person's experience with cognitive decline is unique, an optimal method would utilize an adaptive neural network algorithm to provide intuitive assistance during memory therapy. Such a method would evolve with the user's cognitive abilities as they change over time, continually adjusting the difficulty level of memory therapy to ensure sustained user engagement.

The method described in the present disclosure utilizes an adaptive algorithm to create multi-sensory stimulation for conducting personalized memory therapy on a mobile computing device, for the benefit of people with cognitive decline.

### SUMMARY OF THE INVENTION

This summary is provided to introduce a selection of concepts in simplified form that are further described herein. This summary is not intended to identify key features or essential features of the claimed subject matter, nor is it intended to be used as an aid in determining the scope of the claimed subject matter. Emerging research in neuroscience at leading universities around the world reports that sensory stimulation with specific vibrational frequencies can improve memory by increasing dopamine levels in the brain. Within the hippocampus, the increased dopamine levels facilitate the formation of associative memory, spatial learning and synaptic plasticity. Recent studies involving patients with Alzheimer's disease report that cognitive training using photographic and musical stimulation results in significant improvements in memory function.

In various embodiments of the provided disclosure, a haptic pattern generates vibrotactile stimulation on a mobile computing device for the targeted activation of sensory neurons in fingertips, causing an increase in dopamine levels within the brain and improving the efficacy of memory therapy. Synchronization of the haptic pattern with music produced by speakers within the mobile computing device or external headphones, creates a synergistic sensory effect that further increases dopamine levels and provides additional efficacy for memory therapy. A facial recognition algorithm analyzes digital images and videos to identify familiar individuals by name, relationship and event. In an embodiment, the algorithm utilizes the data from facial recognition to generate personalized cognitive challenges with images and video, while vibrotactile stimulation with synchronized music reinforces learning and recall during memory therapy. Additional features will become apparent herein.

### TECHNICAL FIELD OF THE INVENTION

This invention relates to a method for conducting memory therapy on a mobile computing device. Particularly, this invention relates to conducting memory therapy on a mobile computing device using facial recognition. More particularly, the invention relates to a method for conducting memory therapy on a mobile computing device using facial recognition and haptic stimulation. Specifically, the invention relates to a novel technique for conducting memory therapy on a mobile computing device using facial recognition and vibrotactile stimulation with synchronized music. The invention is also further applicable to conducting cognitive training on a mobile computing device using facial recognition of digital images and videos to identify familiar individuals, who are then presented during memory therapy using vibrotactile stimulation with synchronized music to reinforce learning and recall.

### BRIEF DESCRIPTION OF DRAWINGS

The teachings of the embodiments can be readily understood by considering the following detailed description in conjunction with the accompanying drawings.

FIG. 1 is a flowchart of the method for conducting memory therapy, detailing the phases from user registration through multi-sensory stimulation, in accordance with an embodiment.

FIG. 2 is an overview of the method for subject identification by name and relationship, according to an embodiment.

FIG. 3 illustrates how the method for memory therapy utilizes facial recognition for subject identification, in accordance with one embodiment.

FIG. 4 is a flowchart that illustrates the method for inputting an image into the memory therapy database, in accordance with one embodiment.

FIG. 5 is an example view of the 3D relationship matrix, with a block diagram detailing the method for interactive relationship visualization, according to an embodiment.

FIG. 6 illustrates the method for dynamic linking of family members, in accordance with one embodiment.

FIG. 7 is a flowchart which depicts an interactive memory challenge, detailing the method for using vibrotactile music to reinforce memory, according to an embodiment.

FIG. 8 is an overview of the method for conducting a cognitive challenge using facial recognition, in accordance with one embodiment.

FIG. 9 illustrates the method of locating an individual in a selection of images during a cognitive challenge, according to an embodiment.

FIG. 10 is an example of a multiple-choice cognitive challenge during memory therapy, in accordance with one embodiment.

FIG. 11 illustrates the method of using facial recognition to identify subjects by name, relationship and event during memory therapy, according to an embodiment.

FIG. 12 is an overview of data analytics being utilized to track performance for memory therapy, detailing the method for data collection and processing, in accordance with one embodiment.

FIG. 13 illustrates the percentage increase or decrease of performance results for identifying individual family members, according to an embodiment.

FIG. 14 is an example of data analytics for memory therapy, detailing several different types of infographics, in accordance with one embodiment.

FIG. 15 illustrates synchronizing a haptic pattern with an audio source to create vibrotactile music stimulation, according to an embodiment.

FIG. 16 is an overview of sensory neuron activation with vibrotactile stimulation at gamma frequencies, in accordance with one embodiment.

FIG. 17 illustrates the tactile neuron density in various sections of fingertips, and the contact points for vibrotactile stimulation, according to an embodiment.

FIG. 18 is an example view of vibrotactile music stimulation being used to reinforce facial recognition during memory therapy, according to an embodiment.

FIG. 19 illustrates a playlist of preview images for family members being synchronized with vibrotactile music stimulation, in accordance with one embodiment.

FIG. 20 is a flowchart that details the method for autoplaying multi-sensory stimulation to augment memory therapy, in accordance with one embodiment.

FIG. 21 is an example view of intuitive autoplay mode, with a block diagram detailing the method for user-generated preferences utilizing a dynamic playlist, in accordance with one embodiment.

FIG. 22 is an overview of the method for source selection with audio input, utilizing a block diagram to detail how the algorithm is synchronized with haptic output, according to an embodiment.

FIG. 23 illustrates user-adjustable controls for changing the balance of haptic stimulation with audio output during memory therapy, in accordance with one embodiment.

### DETAILED DESCRIPTION OF THE INVENTION

In the following description of embodiments, numerous specific details are set forth in order to provide a more thorough understanding. However, note that the embodiments may be practiced without one or more of these specific details. In other instances, well-known features have not been described in detail to avoid unnecessarily complicating the description. Embodiments are described herein with reference to the figures where like reference numbers indicate identical or functionally similar elements. Also in the figures, the left most digits of each reference number correspond with the figure in which the reference number is first used.

Embodiments relate to a method of conducting memory therapy using facial recognition with vibrotactile stimulation and synchronized music. Emerging studies in neuroscience show that sensory stimulation with specific vibrational frequencies can augment learning, improve cognition and enhance synaptic plasticity. A haptic pattern generates vibrotactile stimulation on a mobile computing device for the targeted activation of sensory neurons in fingertips, causing an increase in dopamine levels within the brain and improving the efficacy of memory therapy. Synchronization of the haptic pattern with music produced by speakers within the mobile computing device or external headphones, creates a synergistic sensory effect that further increases dopamine levels and provides additional efficacy for learning and recall. A facial recognition algorithm analyzes a digital image or video to identify familiar individuals by name, relationship and event. The algorithm utilizes the data from facial recognition to generate personalized cognitive challenges, and continually adjusts the difficulty level to ensure sustained user engagement, while vibrotactile stimulation with synchronized music reinforces learning and recall during memory therapy.

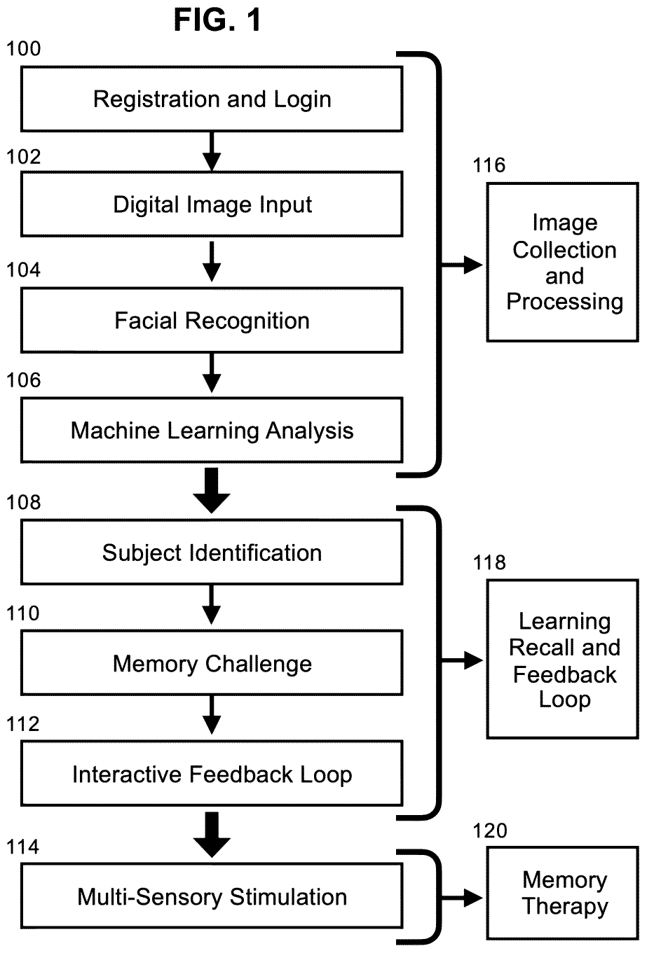

FIG. 1 is a flowchart that illustrates the method for conducting memory therapy, detailing the phases from user registration through multi-sensory stimulation, in accordance with an embodiment. The registration and login 100 may include a data input screen on the mobile computing device, or a data input screen that is accessible through an internet browser on a remote computer. Registration data may reside locally on the mobile computing device and/or on a remote server connected to the internet. Inputting digital images or videos 102 may be accomplished by transferring files from a local library on the mobile computing device or by linking files residing on a remote server. Digital images or videos stored on social media websites can be linked to the memory therapy database for processing and direct access during cognitive training. Physical photographs can be scanned by an external image capture device or by using the camera within a mobile computing device. The facial recognition algorithm 104 uses spatial analysis and matching to build a visual model of the user's relationships with familiar individuals. This method leverages a convolutional neural network 310 to generate cognitive challenges regarding familiar individuals that are based on data from facial recognition. The neural network algorithm can reside locally on the mobile computing device, and/or be accessed on a remote server connected to the internet. The prediction and model aggregation engine 308 detects eye and face geometry 304 to identify individuals by name, relationship and the event depicted 508. The machine learning analysis algorithm 106 manages data from facial recognition to build a relational database that will be used throughout all phases of memory therapy and cognitive training. The database may be stored locally on the mobile computing device, and/or be accessed on a remote server connected to the internet. The subject identification data will be used to create a personalized memory challenge 110 and for cognitive training. In an embodiment, the method for conducting memory therapy will utilize the neural network algorithm 310 to continuously monitor and adapt with the user's cognitive abilities to ensure sustained engagement throughout all phases of cognitive training. The interactive feedback loop 112 leverages the adaptive capabilities of the neural network algorithm 310 to ensure that memory challenges continually evolve with user abilities. As reinforcement for the feedback loop, multi-sensory stimulation 114 can be used as a reward for correct answers, and also to strengthen associative memories when users are performing at sub-standard levels. The sensory stimulation may be comprised of a haptic pattern, music synchronized with the haptic pattern, video graphics, and/or a combination of one or more sensory stimuli.

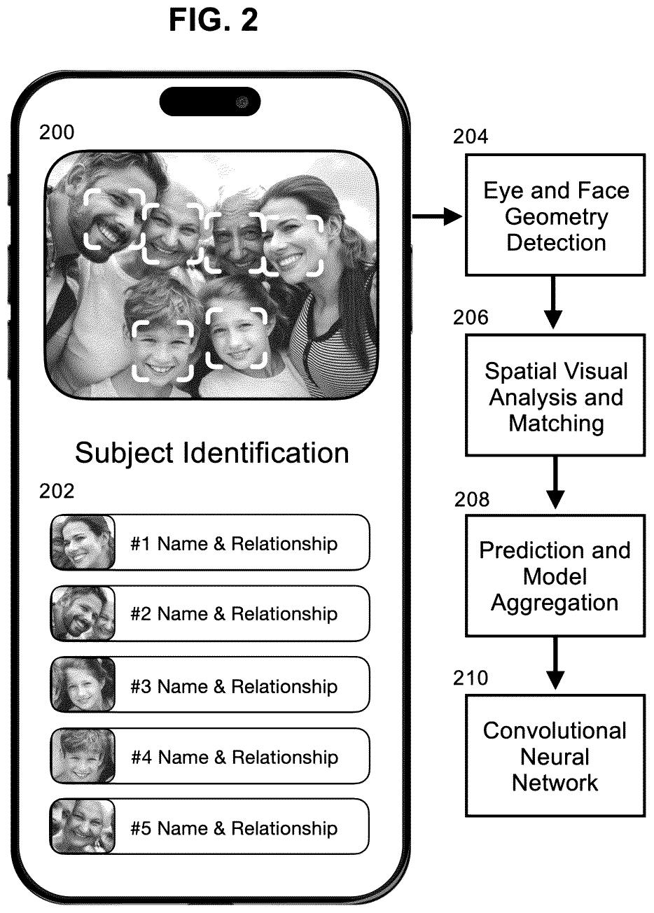

FIG. 2 depicts the method for subject identification by name and relationship, according to an embodiment. A digital image or video is displayed 200 on the screen of a mobile computing device with identification crop marks positioned over the faces of familiar individuals. Each individual is displayed as a thumbnail image 202, and identified by name and relationship in a scrolling menu that is continuously refreshed as new digital images and videos transition into view. By selecting a menu item, additional information about that individual is displayed, including age, location of residence and a description of the event depicted. Eye and face geometry 204 is used to identify the unique physical characteristics of each person, while spatial visual analysis 206 matches individuals, and groups them together in personalized memory challenges 110 using related digital images and videos. The accuracy of facial recognition is enhanced by using prediction and model aggregation 208, which is managed by the convolutional neural network algorithm 210. To reinforce memory function during subject identification, vibrotactile stimulation with synchronized music is generated 704 for the targeted activation of fingertip neurons, and the subsequent increase of dopamine levels in the brain.

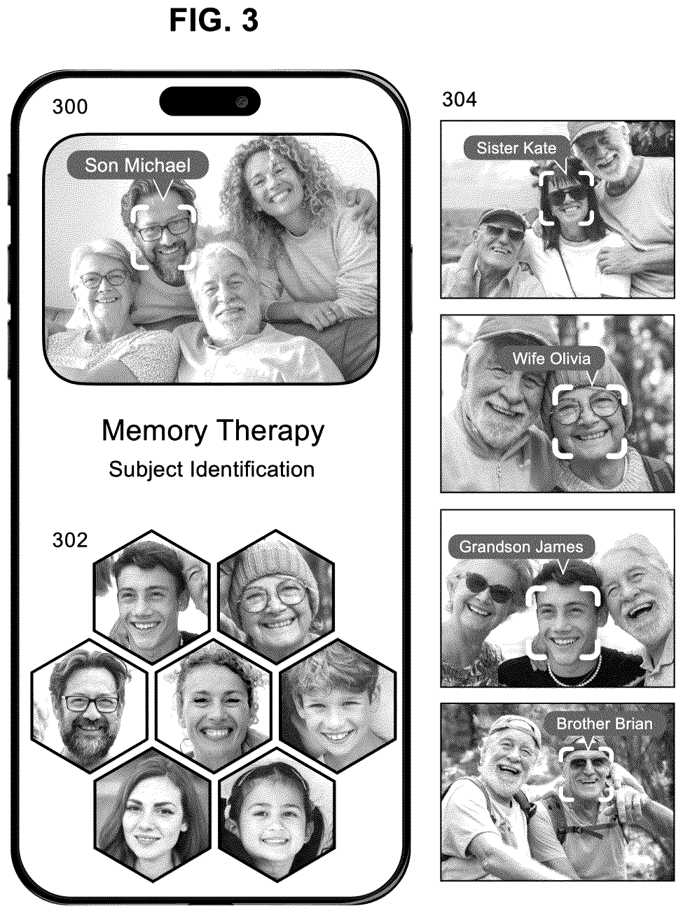

FIG. 3 illustrates how the method for conducting memory therapy uses facial recognition for subject identification, in accordance with one embodiment. A digital image or video is displayed 300, having identification crop marks positioned over the face of a familiar individual, with that person's name being displayed in the screen of the mobile computing device. In one embodiment the name is displayed in white typeface with a dark background border. In another embodiment the typeface is dark with a light background border. The name may be displayed above the identified individual, or below it, and in another embodiment the name may be to the left or right from the individual's face. A grouping of thumbnail images 302 is displayed on the screen, and acts as a selectable menu having buttons that display the names of other individuals 304 that appear in the digital images or videos. To reinforce memory function during subject identification, vibrotactile stimulation with synchronized music is generated 704 for the targeted activation of fingertip neurons, and the subsequent increase of dopamine levels in the brain.

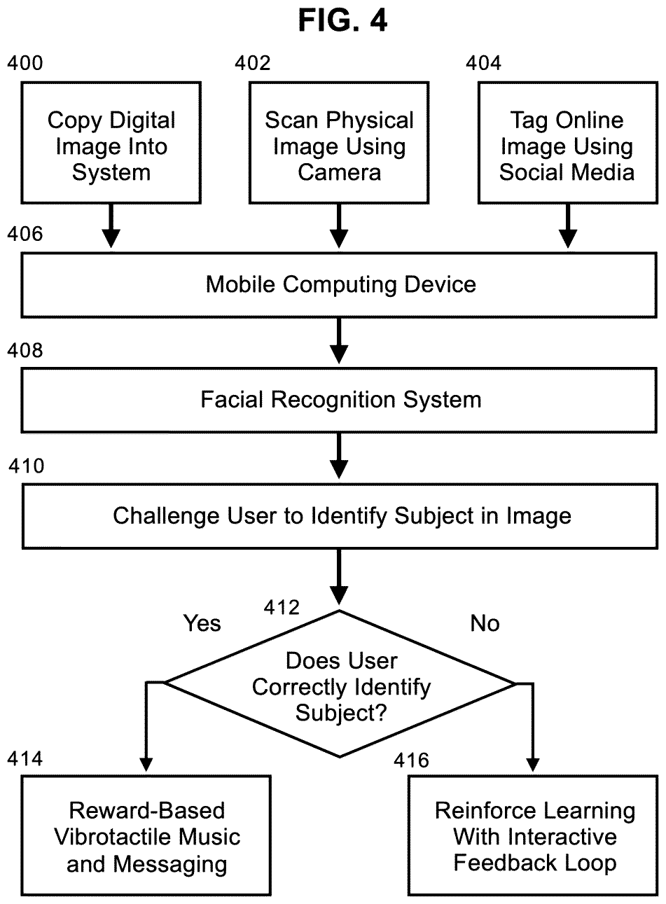

FIG. 4 is a flowchart that illustrates the method for inputting a digital image or video into the memory therapy database, in accordance with one embodiment. Ingestion of a digital image or video 400 may be accomplished by transferring files from a local library on the mobile computing device via copying and pasting the file into the memory therapy database. In another embodiment, the file can be linked from a local database or linked to a file residing on a remote server connected to the internet. Physical photographs 402 can be scanned using an external image capture device or by using the camera within the mobile computing device. Digital images or videos 404 stored on social media websites can be linked to the memory therapy database for processing and direct access during cognitive training. In one embodiment, the mobile computing device 406 will store a digital file of the image or video, and in another embodiment a digital image or video will be tagged or linked to a remote server accessible on the internet. The facial recognition algorithm 406 accesses the database containing the digital image or video, and creates a cognitive challenge 410 for the user to identify a familiar individual by name, relationship and a description of the event depicted. A computer algorithm 412 determines whether the user has correctly identified the individual, and either rewards the user with vibrotactile music and positive messaging 414, or reinforces learning via the interactive feedback loop 416.

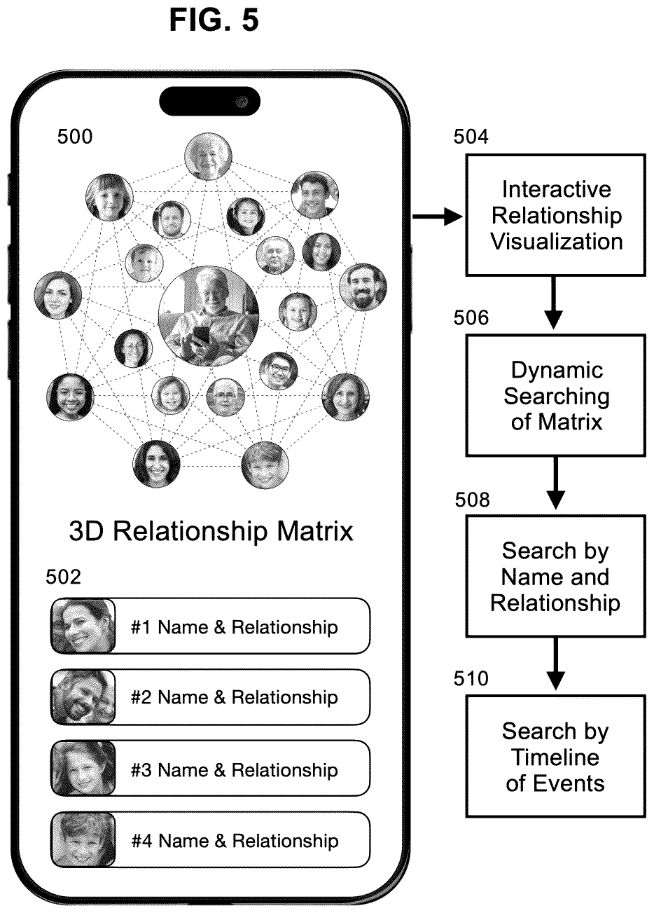

FIG. 5 is an example view of the 3D relationship matrix, with a block diagram detailing the method for interactive relationship visualization, according to an embodiment. Thumbnail images of familiar individuals known to the user are displayed 500 in a three-dimensional matrix which operates as a selectable menu. A dynamic graphics engine renders the 3D matrix in real-time and enables the user to scroll through the menu in a three-dimensional space. Selecting a particular thumbnail image will reveal an expanded data field 502 which displays additional information about the individual, including age, location of residence and a description of the event depicted. The method for interactive relationship visualization 504 enables dynamic searching of the 3D matrix 506 by name and relationship 508, and also allows for searching via an interactive timeline of events 510.

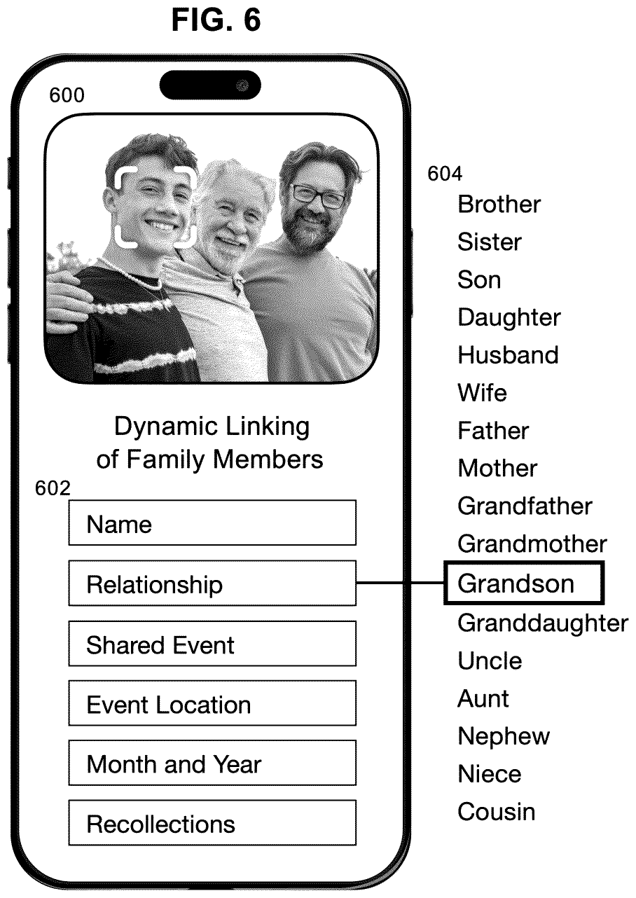

FIG. 6 illustrates the method for dynamic linking of family members, in accordance with one embodiment. Upon ingestion into the database, each individual identified in a digital image or video 600, will appear with crop marks around their face. Data fields 602 will be displayed for the inputting of personal information including name, relationship, event, location, month/year, and a brief description of the event. The relational database will link individuals who are members of the same family, for the purpose of auto-filling data fields and batch processing of related images. When the facial recognition algorithm identifies an individual who is present in multiple digital images or videos, it will present details known about that individual including name and familial relationship, as a highlighted list for each data field, for the purpose of expediting the labeling of multiple digital images and videos.

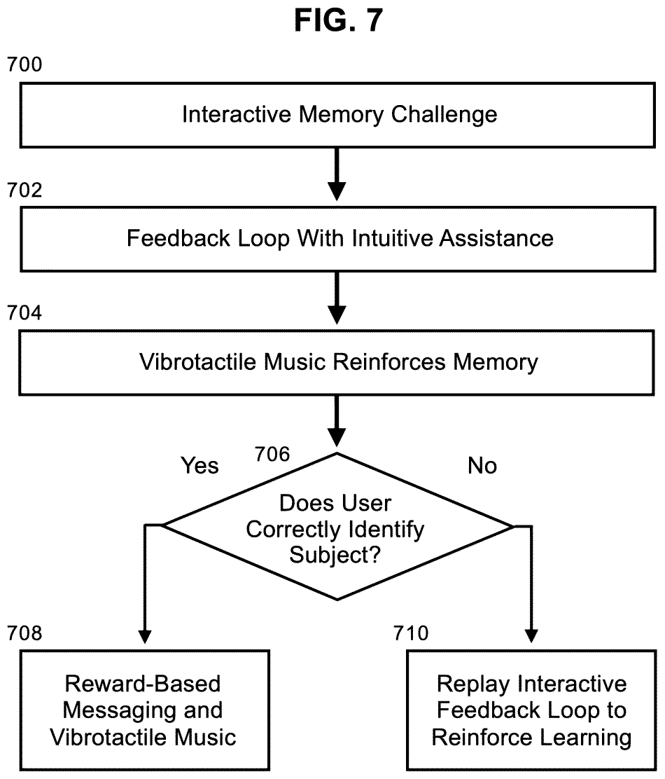

FIG. 7 is a flowchart that depicts an interactive memory challenge, detailing the method for using vibrotactile stimulation with synchronized music to reinforce memory, according to an embodiment. In one embodiment, the interactive memory challenge 700 may be to identify a familiar individual by name and relationship. In another embodiment, the challenge may be to identify the event or location in which the individual is depicted. In another embodiment, the challenge may include locating the individual's face in a selection of numerous digital images or videos 800. Another embodiment may be a multiple-choice challenge to identify a particular individual in a group of people by name, relationship or description of the event depicted. The feedback loop 702 reinforces memory therapy using vibrotactile stimulation with synchronized music 704, and utilizes the neural network algorithm 706 to determine whether the user has correctly identified an individual in the digital image or video. The convolutional neural network algorithm 210 evaluates the results of the challenge, and either rewards the user with vibrotactile music and positive messaging 708, or replays the interactive feedback loop 710 to reinforce learning.

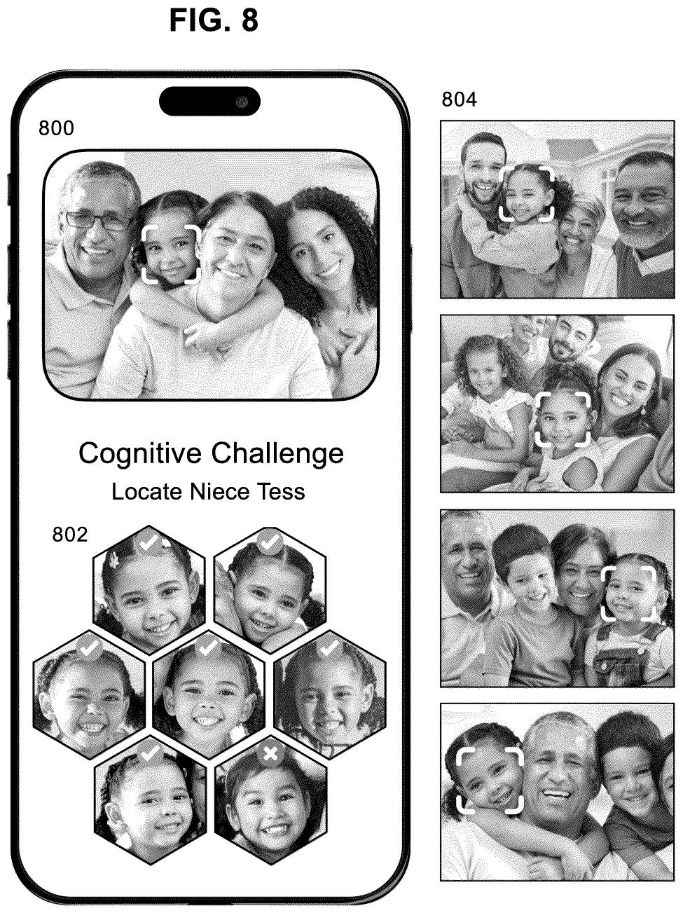

FIG. 8 is an overview of the method for conducting a cognitive challenge using facial recognition, according to an embodiment. A digital image or video is displayed 800, and the user is challenged to locate a specific individual within a group of people by touching an area surrounding the individual's face. Upon selection, a crop mark will appear around the perimeter of the individual's face, and either a check mark or an “X” mark will appear to denote whether the user has selected the correct individual. A thumbnail image will appear on the screen to display the results of each successive challenge. In one embodiment, the user swipes images or videos at their own pace, and in another embodiment the computer algorithm controls the timing of images being presented. Results from the cognitive challenge are displayed dynamically, and are continuously updated to reflect the percentage of correct answers during a particular cognitive challenge.

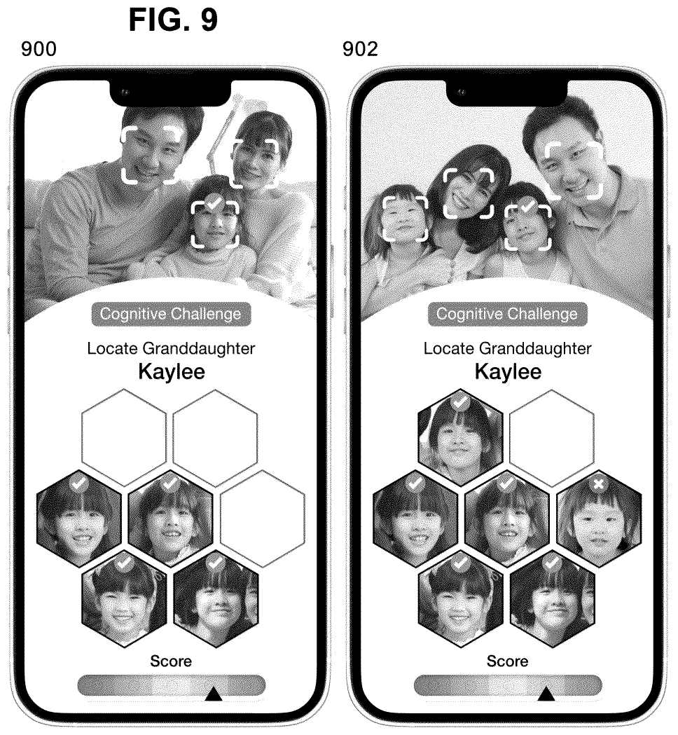

FIG. 9 illustrates the method of locating an individual in a selection of images during a cognitive challenge, according to an embodiment. Throughout multiple cognitive challenges 900, thumbnail images of the identified individual will be displayed in order. A check mark or an “X” mark 902 will appear above each thumbnail image to denote whether the user has selected the correct individual. Results from the cognitive challenge are displayed dynamically and are continuously updated to reflect the percentage of correct answers during a particular cognitive challenge. In one embodiment, the user swipes images or videos at their own pace, and in another embodiment the computer algorithm controls the timing of images being presented.

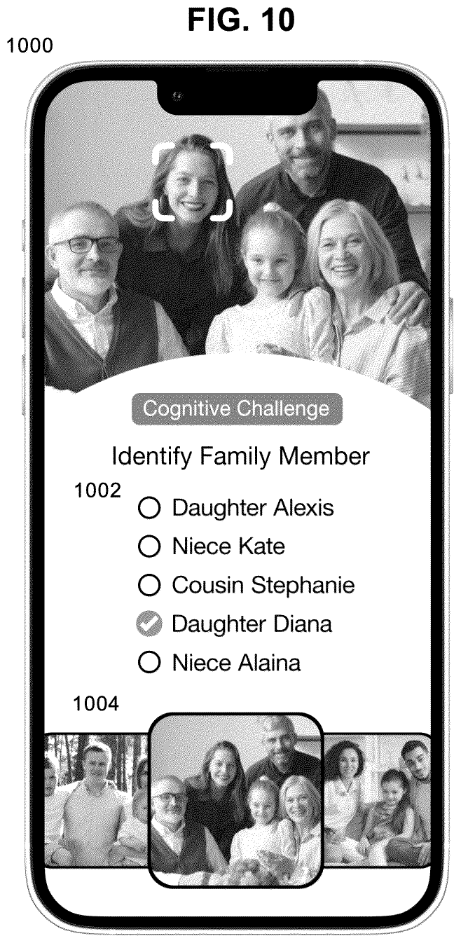

FIG. 10 is an example of a multiple-choice cognitive challenge during memory therapy, in accordance with one embodiment. A digital image or video is presented 1000, and crop marks are displayed around one particular individual's face. The user is challenged to identify that individual within the group of people by correctly selecting one response from multiple potential choices 1002. Upon selection, either a check mark or an “X” mark will appear next to the choice the user has selected to denote a correct or incorrect answer. In one embodiment, the names and relationships of potential family members are displayed. In another embodiment, events and/or locations are used as potential choices. In either scenario, there is one correct answer and multiple incorrect answers that may or may not include the individuals appearing in the digital image or video. Thumbnail images 1004 are arranged in a dynamic carousel menu to control the sequence of cognitive challenges being presented. The dynamic menu can be controlled by the user, or may be set to play automatically in an autonomous slideshow mode of operation.

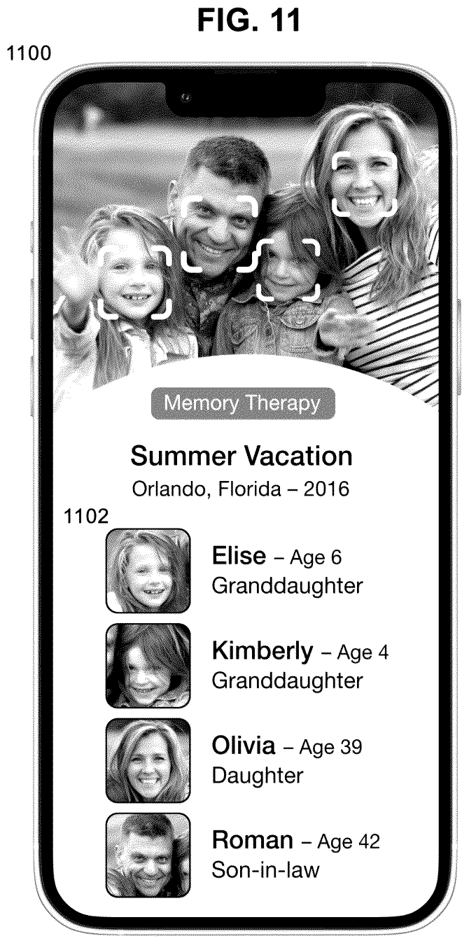

FIG. 11 illustrates the method for using facial recognition to identify subjects by name, relationship and event during memory therapy, according to an embodiment. A digital image or video is displayed 1100 on the screen of a mobile computing device with identification crop marks positioned over the faces of familiar individuals. Each individual is presented as a thumbnail image 1102, and identified by name and relationship in a scrolling menu that is continuously refreshed as new digital images and videos transition into view. By selecting a menu item, additional information about that individual is displayed, including age, location of residence and a description of the event depicted. To reinforce memory function during subject identification, vibrotactile stimulation with synchronized music is generated 1800 for the targeted activation of fingertip neurons, and the subsequent increase of dopamine levels in the brain.

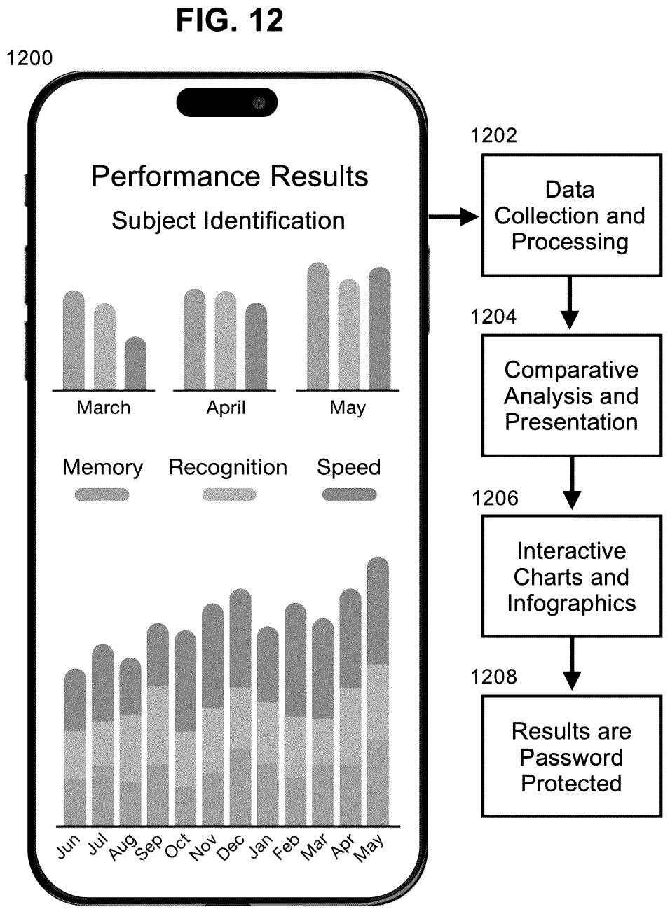

FIG. 12 is an overview of data analytics that track performance for memory therapy, detailing the method for data collection and processing, in accordance with one embodiment. The convolutional neural network algorithm 210 stores the results from cognitive challenges in a relational database, which is then used to display various performance metrics 1200, which provide a detailed overview of memory function over time. In one embodiment, metrics for performance include recognition, speed and overall memory ability, each presented as detailed infographics in colors that correspond to the various associated categories. The convolutional neural network algorithm performs data collection and processing 1202 for the comparative analysis 1204, and presentation of interactive charts and infographics 1206, which are password protected 1208, for secure dissemination to approved third-parties for review. In one embodiment, the database containing performance metrics is stored locally in the mobile computing device. In another embodiment, the database of performance metrics is stored on a remote computer server connected to the internet.

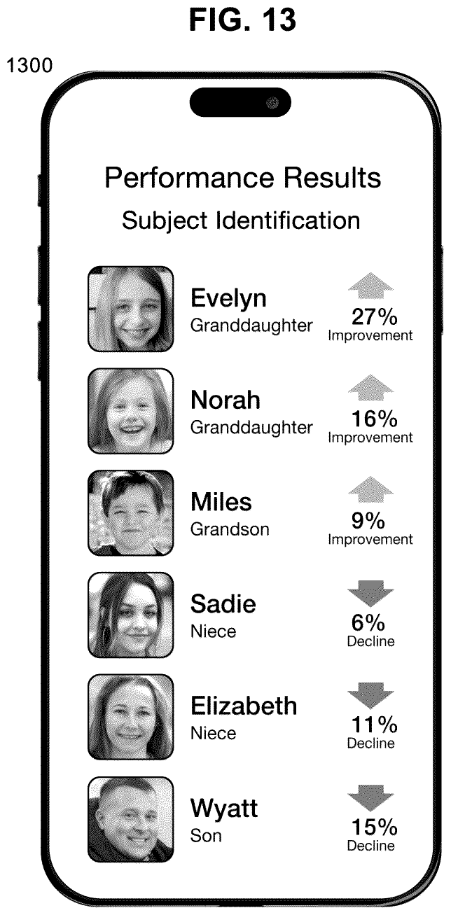

FIG. 13 illustrates the percentage increase or decrease of performance results for identifying individual family members, according to an embodiment. Results from cognitive challenges are stored in the relational database and used to present a detailed overview of a user's performance for identifying familiar individuals 1300. Thumbnail images for specific individuals are displayed by name and relationship, with a corresponding percentage value denoting an increase or decrease in recognition performance. The performance results are also presented with graphical arrows in the colors of green for increased performance and red for a decline in performance.

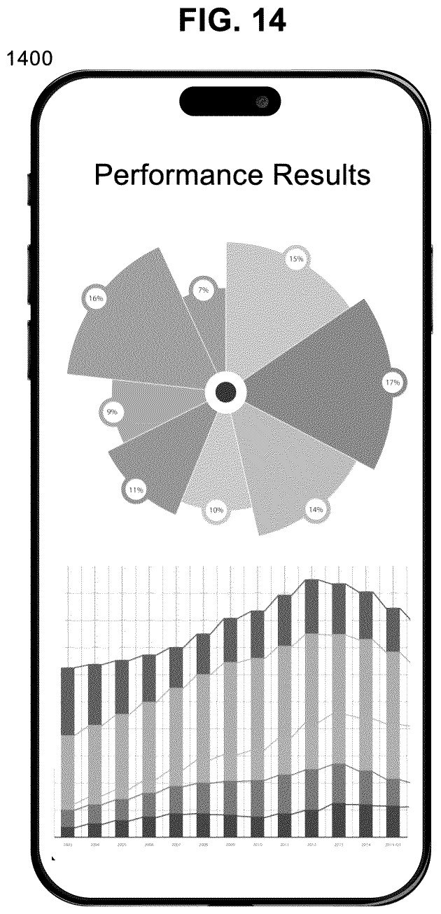

FIG. 14 is an example of data analytics for memory therapy, detailing several different types of infographics, in accordance with one embodiment. Animated charts and graphs 1400 present data analytics in a visually dynamic and engaging manner, displaying detailed performance metrics in a variety of colors that correspond with challenge categories. Animated infographics may include a bar chart, pie graph, circular chart, Venn diagram, alphanumeric chart, word cloud, graphical icons, and combinations of letters and/or numbers.

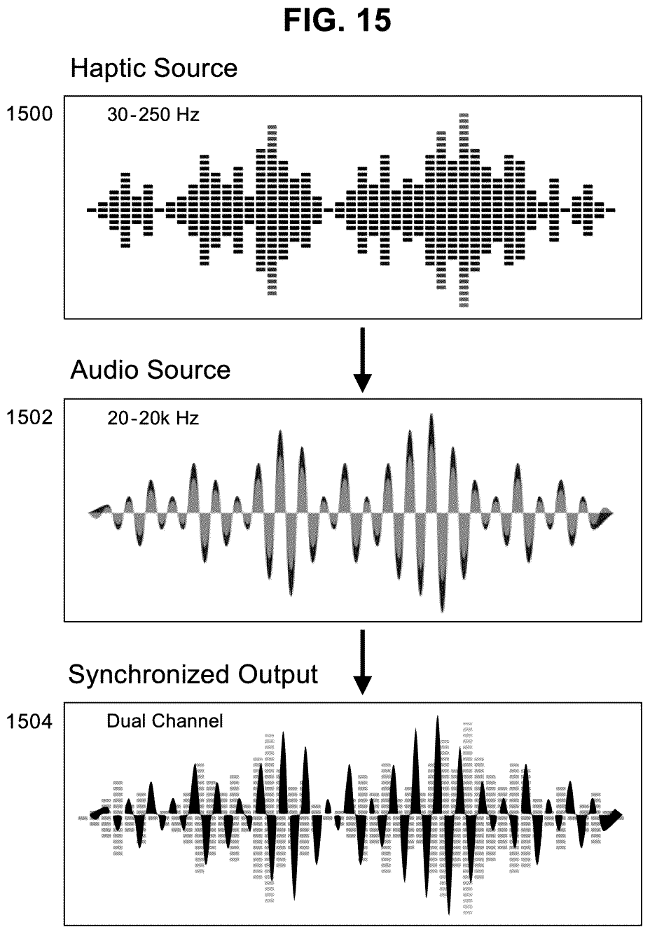

FIG. 15 illustrates synchronizing a haptic pattern with an audio source to create vibrotactile music stimulation, according to an embodiment. A haptic pattern 1500 generates vibrotactile stimulation on a mobile computing device for the targeted activation of sensory neurons in fingertips 1708, causing an increase in dopamine levels within the brain and improving the efficacy of memory therapy. Synchronization of the haptic pattern with music 1502 produced by speakers within the mobile computing device or external headphones, creates a synergistic sensory effect 1504 that further increases dopamine levels for improved memory function. Emerging research shows that vibrotactile stimulation at gamma frequencies of 30 to 140 Hz can improve memory function by increasing dopamine levels in the brain. The method for conducting memory therapy utilizes an acoustic algorithm to generate vibrotactile stimulation on the mobile computing device at frequencies of 30 to 140 Hz. The vibrotactile stimulation is synchronized with the audio source file being played at frequencies of 20 to 20,000 Hz on speakers contained within the mobile computing device, and/or external headphones. In one embodiment, a library of pre-composed audio files and haptic patterns are provided as a playlist within the mobile computing device 2202, the audio files and haptic patterns having been previously synchronized for producing a greater sensory response together, than they would as separate sensory stimuli. In another embodiment, the haptic pattern is generated from an audio file residing within the mobile computing device and/or located on an external online computer server 2206. The haptic pattern may be generated using an algorithm residing within the mobile computing device and/or located on an external online computer server. The resulting haptic pattern is then synchronized with the audio playback 2214 to generate multi-sensory stimulation for the reinforcement of learning and recall during memory therapy. In another embodiment, the vibrotactile stimulation with synchronized audio playback may be augmented with video graphics displayed concurrently on a screen contained within the mobile computing device and/or an external display monitor, the video graphics being synchronized with the multi-sensory stimulation to further reinforce learning and recall during memory therapy.

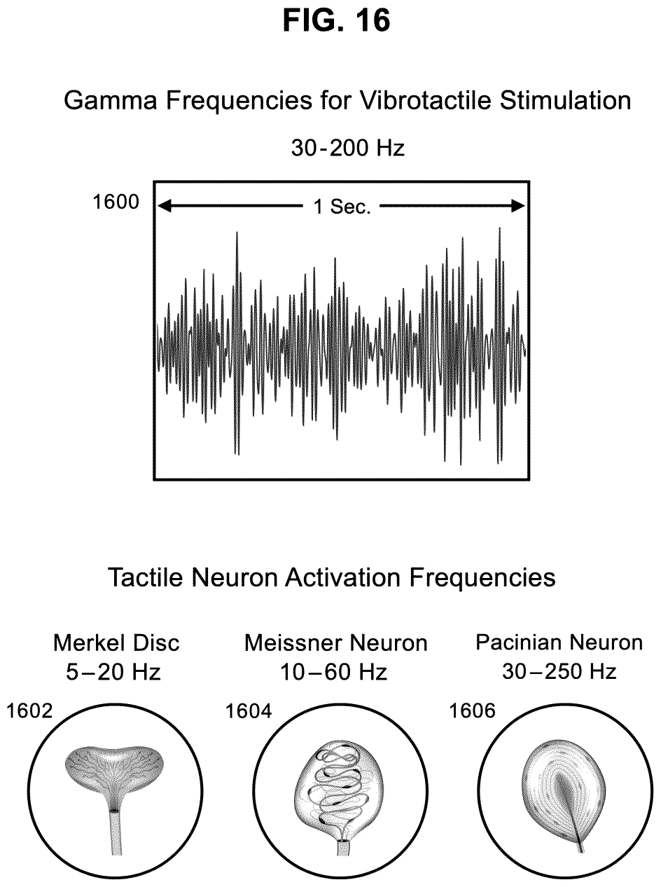

FIG. 16 is an overview of sensory neuron activation using vibrotactile stimulation at frequencies of 30 to 140 Hz 1600, in accordance with one embodiment. This gamma wave frequency range corresponds with tactile neurons including the Merkel disc 1602, Meissner corpuscle 1604 and Pacinian corpuscle 1606.

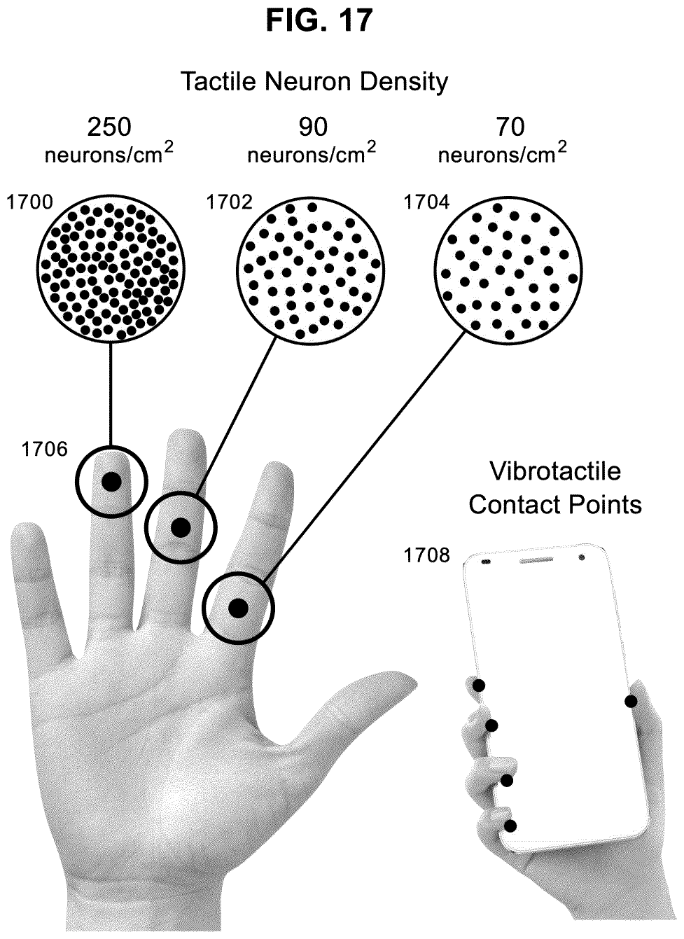

FIG. 17 depicts the tactile neuron density in various sections of fingertips and the contact points for vibrotactile stimulation, according to an embodiment. Fingertips 1706 contain some of the highest densities of sensory neurons in the human body, and are an ideal location to conduct vibrotactile stimulation. Holding a mobile computing device in one hand with the screen facing forward 1708 provides direct contact with the fingertips 1700, the mid-points of fingers 1702 and the base of fingers 1704. These contact points have been shown through clinical studies to offer an effective mode of stimulating tactile neurons for increasing the level of dopamine in the brain, and subsequently improving memory function.

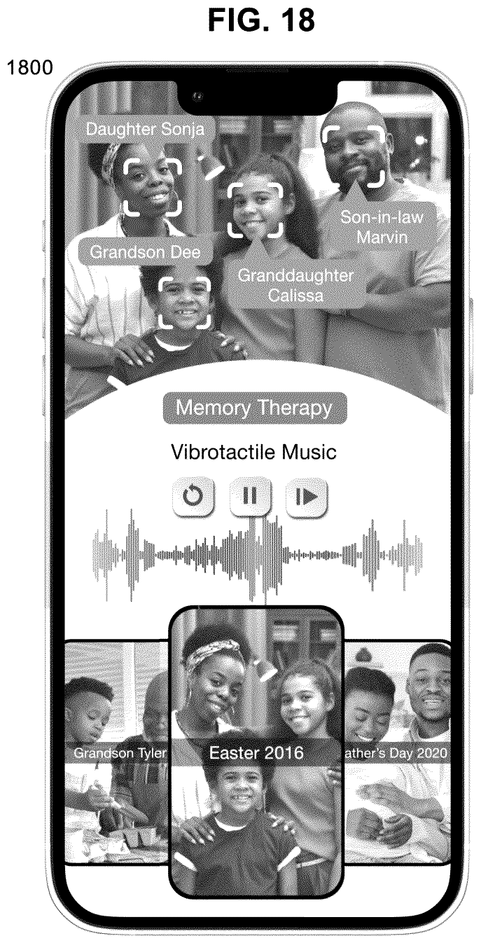

FIG. 18 is an example view of vibrotactile stimulation with synchronized music being used to reinforce facial recognition during memory therapy, according to an embodiment. Vibrotactile stimulation with synchronized music is utilized for the interactive feedback loop 416, and provides reward-based reinforcement during memory therapy 414. When multi-sensory stimulation is generated while concurrently displaying the names and relationships of familiar individuals 1800, synaptic plasticity is enhanced and new associative memories are formed by the hippocampus. In one embodiment, a library of pre-composed audio files and haptic patterns are provided as a playlist within the mobile computing device, the audio files and haptic patterns having been previously synchronized for producing a greater sensory response together, than they would as separate sensory stimuli. In another embodiment, the haptic pattern is generated from an audio file residing within the mobile computing device and/or located on an external online computer server 2200. The algorithm may reside within the mobile computing device and/or be located on an external online computer server. The resulting haptic pattern is then synchronized with the audio playback 2214 to generate multi-sensory stimulation for the reinforcement of learning and recall during memory therapy. In another embodiment, the vibrotactile stimulation with synchronized audio playback may be augmented with video graphics displayed concurrently on a screen contained within the mobile computing device and/or an external display monitor, said video graphics being synchronized with the multi-sensory stimulation to further reinforce learning and recall during memory therapy.

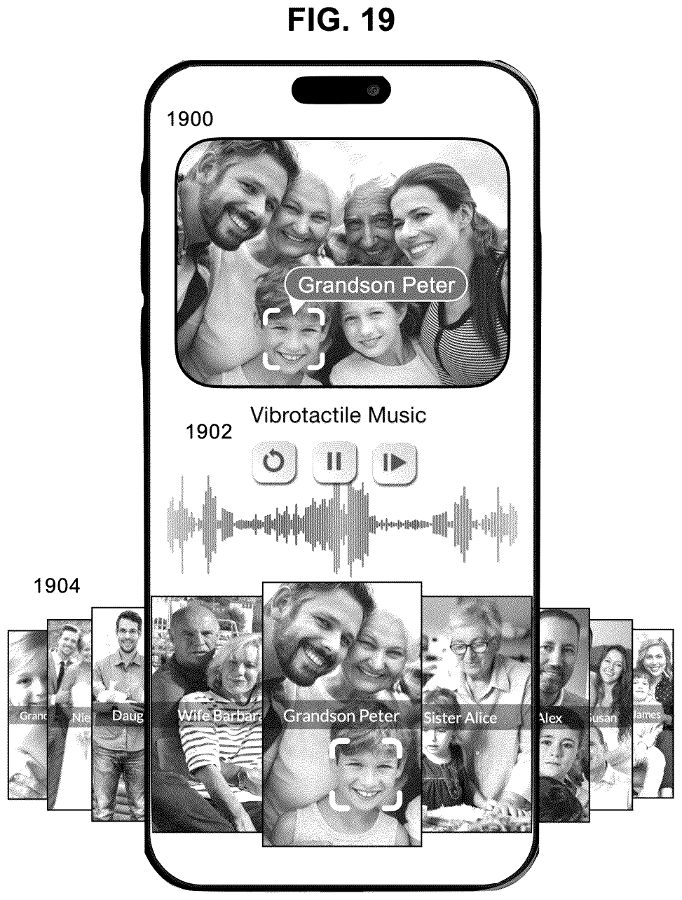

FIG. 19 illustrates a playlist of thumbnail images depicting family members, the memories of whom are reinforced using vibrotactile stimulation with synchronized music, in accordance with one embodiment. A digital image or video is displayed 1900 on the screen of a mobile computing device with identification crop marks positioned over the face of a familiar individual. To reinforce memory function during subject identification, vibrotactile stimulation with synchronized music is generated 1902 for the targeted activation of fingertip neurons, and the subsequent increase of dopamine levels in the brain. A carousel menu of thumbnail previews is displayed 1904, which corresponds with the digital images or videos being identified by facial recognition in the main screen. The vibrotactile music player can be operated with individual buttons that control functions including: play, pause, loop and autoplay. A library of vibrotactile music 2202 can be programmed such that a specific soundtrack can be linked to correspond with a particular digital image or video of a familiar individual. By associating an individual with a specific vibrational soundtrack, associative memories about that individual can be formed and/or reinforced.

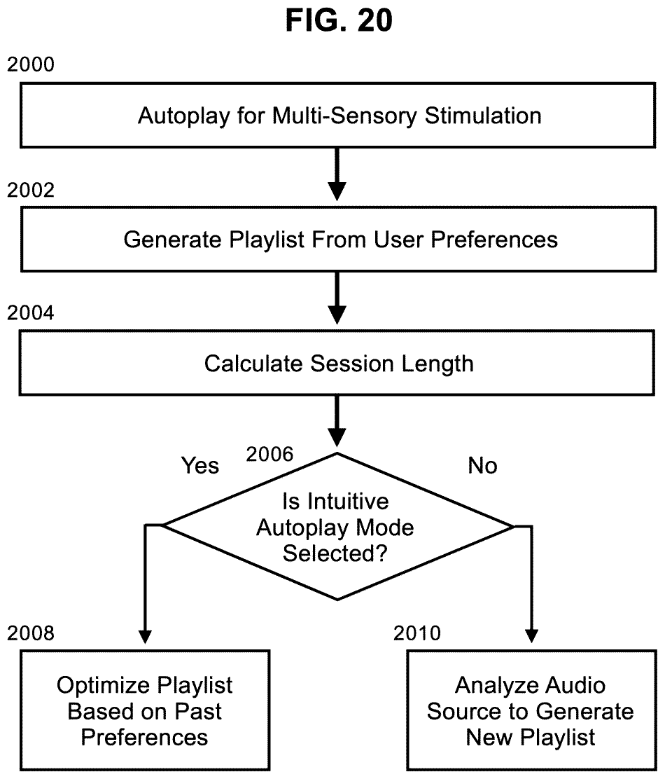

FIG. 20 is a flowchart that details the method for autoplaying multi-sensory stimulation to augment memory therapy, in accordance with one embodiment. During memory therapy, vibrotactile stimulation with synchronized music will be generated to reinforce learning and recall. The multi-sensory stimulation can be programmed to autoplay during cognitive training 2000, such that specific soundtracks will be associated with a particular individual. The neural network algorithm will utilize data from user preferences to generate a playlist of soundtracks 2002 to reinforce memory therapy. Based on historical data from user performance, the neural network algorithm will calculate the appropriate session length 2004 that will optimize learning and recall during cognitive training. When the autoplay mode is selected 2006, the computer algorithm will either optimize the playlist based on past user preferences 2008, or analyze the audio source to generate a new playlist 2010.

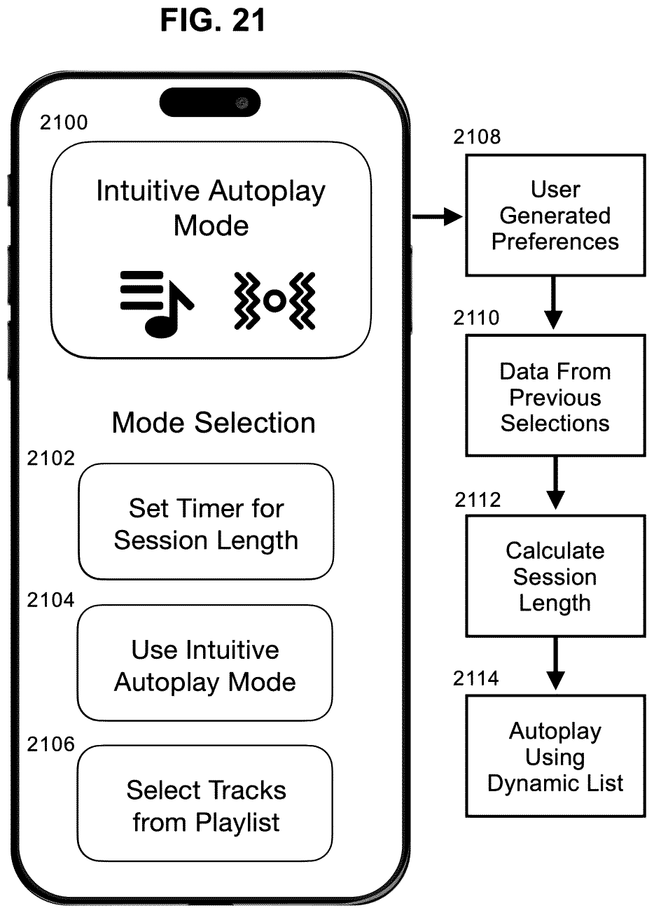

FIG. 21 illustrates an example of intuitive autoplay mode, with a block diagram detailing the method for integrating user-generated preferences with a dynamic playlist, in accordance with one embodiment. Intuitive autoplay mode 2100 will engage the neural network algorithm to generate a playlist of vibrotactile stimulation with synchronized music that corresponds with a particular memory therapy session. In one embodiment, the algorithm will offer the options to set a timer for session length 2102, use autoplay mode 2104, or manually select soundtracks from the playlist 2106. The neural network algorithm 2108 utilizes data from previous sessions 2110, to calculate the session length 2112, and initiate autoplay mode using a dynamic list 2114 of vibrotactile soundtracks from the library.

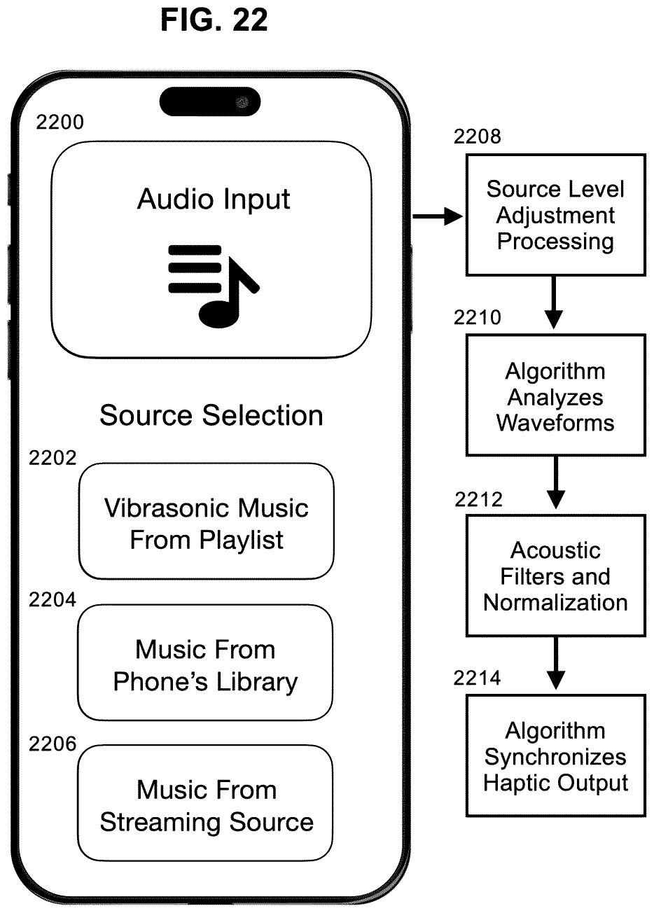

FIG. 22 is an overview of the method for selecting audio input source, while utilizing a block diagram to detail how the algorithm is synchronized with haptic output, according to an embodiment. Vibrotactile stimulation with synchronized music can be generated from multiple input sources 2200. In one embodiment, haptic files and audio source files are contained within a library stored locally, in a playlist within the memory therapy database 2202. In another embodiment, music stored in a local library on the mobile computing device 2204, is used by the neural network algorithm to generate haptic signals resulting in vibrotactile stimulation with synchronized music. In another embodiment, music from a streaming service on a remote internet server 2206 is used by the neural network algorithm to generate haptic signals resulting in vibrotactile stimulation with synchronized music. The neural network algorithm manages source level adjustment processing 2208, while an algorithm analyzes acoustic waveforms 2210 to generate the haptic patterns. The neural network algorithm utilizes acoustic filters and performs source normalization 2212, enabling the algorithm to generate a synchronized haptic pattern 2214.

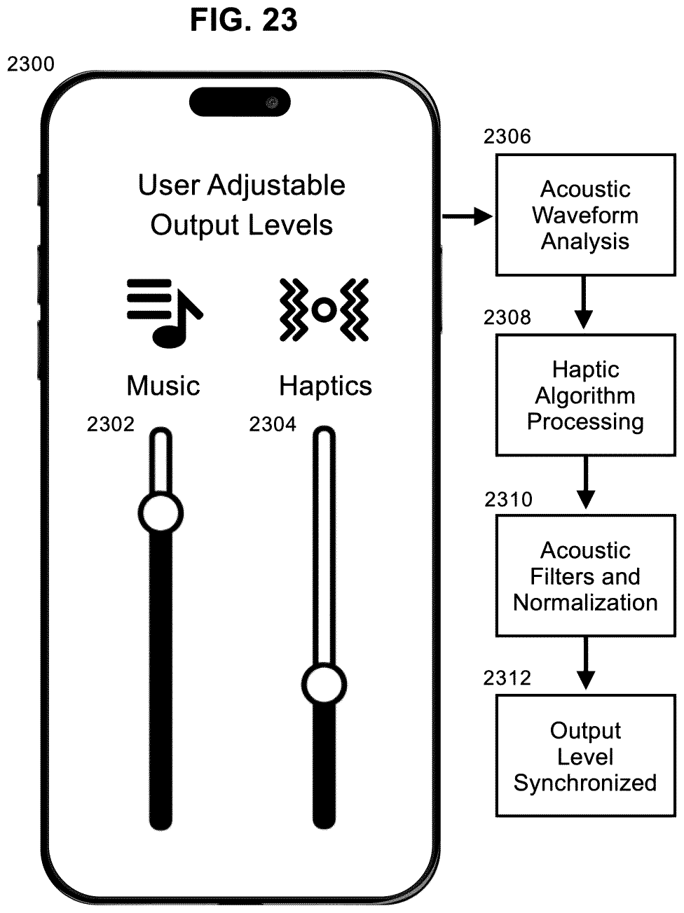

FIG. 23 illustrates user-adjustable controls for changing the balance of haptic stimulation with audio output during memory therapy, in accordance with one embodiment. Haptic patterns and audio source files are synchronized to create multi-sensory stimulation for reinforcing memory therapy. In one embodiment, the output level of each sensory source is fixed 2300. In another embodiment, the output level of music volume 2302, and the output intensity of haptic stimulation 2304 can be adjusted independently using a digital control on the mobile computing device. In one embodiment the control type is a digital slider, and in another embodiment, the control type is a knob or dial interface. In each style of controller, the effect on adjusting the balance of output levels is the same. The neural network algorithm 310 performs acoustic waveform analysis 2306, for haptic algorithm processing 2308 to control the intensity of vibrotactile stimulation. The audio signal is processed with acoustic filters and normalization 2310 to control the synchronization of output levels 2312. In one embodiment, the intensity of vibrotactile stimulation and the volume of music can be adjusted for each individual multi-sensory soundtrack. In another embodiment, the balance and levels of multi-sensory stimulation can be stored as preferences for all soundtracks within a playlist.

What is claimed is:

1. A method for conducting memory therapy on a mobile computing device by generating multi-sensory stimulation to increase dopamine levels in the brain, the method comprising: utilizing a haptic pattern to generate vibrotactile stimulation for the targeted activation of sensory neurons in fingertips, concurrent with the synchronized playback of audio signals through speakers within the mobile computing device and/or external headphones connected physically or wirelessly, the synchronized audio playback creating a synergistic sensory effect with the vibrotactile stimulation to further increase levels of dopamine for the reinforcement of learning and recall during memory therapy.

2. The method of claim 1, wherein a library of pre-composed audio files and haptic patterns are provided as a playlist within the mobile computing device, the audio files and haptic patterns having been previously synchronized for producing a greater sensory response together, than they would as separate sensory stimuli.

3. The method of claim 1, wherein the haptic pattern is generated from an audio file residing within the mobile computing device and/or located on an external online computer server, the haptic pattern being generated using an algorithm residing within the mobile computing device and/or located on an external online computer server, the resulting haptic pattern then being concurrently synchronized with the audio playback to generate multi-sensory stimulation for the reinforcement of learning and recall during memory therapy.

4. The method of claim 1, wherein the vibrotactile stimulation with synchronized audio playback may be augmented with video graphics displayed concurrently on a screen contained within the mobile computing device and/or an external display monitor, said video graphics being synchronized with the multi-sensory stimulation to further reinforce learning and recall during memory therapy.

5. The method of claim 1, wherein the haptic pattern comprises a vibrotactile frequency range of between 30 and 140 Hertz, which corresponds with the frequency range of gamma waves in the human brain.

6. The method of claim 1, wherein the intensity level of vibrotactile stimulation can be adjusted via a digital control on the mobile computing device to customize the balance of combined multi-sensory stimulation being generated.

7. A method for conducting memory therapy on a mobile computing device using facial recognition to identify a familiar individual in a digital image or video, the method comprising: analyzing a selection of digital images or videos stored locally or online for the distinct facial features of a target individual, which is then presented as a cognitive challenge for recalling the identity of the individual by name, relationship and event, for the reinforcement of cognitive training during memory therapy.

8. The method of claim 7, wherein the individual within the digital image or video, having been identified by name, relationship and event, is then presented in a grouping with one or more related digital images or videos of the same individual to reinforce the memories about said individual during cognitive training.

9. The method of claim 7, wherein the memory therapy, utilizing a digital image or video, is conducted with or without sensory stimulation, said sensory stimulation comprising vibrotactile stimulation, audio playback, video graphics, or the combination of two or more stimuli for concurrent multi-sensory stimulation during memory therapy.

10. The method of claim 7, wherein the facial recognition algorithm identifies the individual by name, relationship and event, the individual is then organized into a database by familial structure, such that members of the same family group can be dynamically linked together to create personalized cognitive challenges during memory therapy, the familial database also being accessed when inputting digital images and videos, for facilitating batch processing and auto-filling of data fields.

11. The method of claim 7, wherein the facial recognition algorithm can be accessed and/or configured within the mobile computing device, and/or accessed via a remote online computer server on the internet.

12. The method of claim 7, wherein the source digital image and/or video for facial recognition can be input using the camera within the mobile computing device to capture a still photograph, live-action video, and/or a facsimile of an existing physical photograph for the facilitation of facial recognition during memory therapy.

13. A method for conducting memory therapy on a mobile computing device by utilizing a neural network algorithm to manage all aspects of cognitive training, the method comprising: the facilitation of data input, facial recognition, memory therapy, sensory stimulation, data analysis, user interaction, speech recognition, voice synthesis, and/or communication with external third-parties, for the facilitation of cognitive training during memory therapy.

14. The method of claim 13, wherein the neural network algorithm is operated and accessed within the mobile computing device, and/or is accessed externally via an online machine learning algorithm or generative AI system on the internet, for the facilitation of cognitive training during memory therapy.

15. The method of claim 13, wherein the neural network algorithm continually manages all aspects of conducting memory therapy, said management including adjusting the difficulty level of cognitive challenges as needed to ensure sustained user engagement, while providing interactive assistance in the form of text-based hints, visual cues and/or synthesized speech responses to maintain said user engagement during memory therapy.

16. The method of claim 13, wherein the neural network algorithm renders thumbnail images of individuals previously identified by facial recognition, the thumbnail images creating an interactive 3D menu interface for visualizing and selecting those individuals who will be used during memory therapy and cognitive training.

17. The method of claim 13, wherein the neural network algorithm generates a user-selectable menu using thumbnail images arranged by date and/or event, the timeline menu providing options for interactive scrolling, and/or autonomous slideshow operation during memory therapy.

18. The method of claim 13, wherein the neural network algorithm recognizes spoken commands and initiates the corresponding actions for managing memory therapy and cognitive training, while concurrently generating verbal responses using voice synthesis for real-time conversational interaction to reinforce learning and recall during memory therapy.

19. The method of claim 13, wherein the neural network algorithm monitors user performance during memory therapy to identify any digital images or videos that are not fully recognizable to the user, at which point the algorithm contacts by text message and/or email, other individuals who were present in the digital images or videos, for the purpose of requesting their recollections of the event in the form of text messages, audio recordings and/or video recordings, the recollections then being dynamically linked with other related digital images and/or videos, and presented to the user to reinforce learning and recall during memory therapy.

20. The method of claim 13, wherein the neural network algorithm analyzes user performance during cognitive training and memory therapy, for the purpose of generating data analytics that can be used to track performance over time, said data analytics being displayed as charts and graphs to be shared securely with designated third parties to monitor changes in the user's cognitive function as a result of memory therapy.
